(19) United States
(12) Patent Application Publication
(10) Pub. No.: US 2025/0339453 A1
(43) Pub. Date: Nov. 6, 2025

(54) NUTRITIONAL COMPOSITION FOR IMPROVING GUT MICROBIOTA

(71) Applicant: N.V. NUTRICIA, Zoetermeer (NL)

(72) Inventors: Nana BARTKE, Utrecht (NL); Sebastian TIMS, Utrecht (NL); Christophe LAY, Utrecht (NL); Guus ROESELERS, Utrecht (NL)

(73) Assignee: N.V. NUTRICIA, Zoetermeer (NL)

(21) Appl. No.: 19/269,127

(22) Filed: Jul. 15, 2025

Related U.S. Application Data
(63) Continuation of application No. PCT/EP2024/051955, filed on Jan. 26, 2024.

(30) Foreign Application Priority Data
Jan. 27, 2023 (EP) 23153782.0

(51) Int. Cl. A61K 31/688; A23L 33/00; A61K 31/20; A61K 31/201; A61K 31/202; A61K 35/20; A61P 1/00; A61P 1/14; A61P 31/04

(52) U.S. Cl. CPC A61K 31/688; A23L 33/40; A61K 31/20; A61K 31/201; A61K 31/202; A61K 35/20; A61P 1/00; A61P 1/14; A61P 31/04

(57) ABSTRACT

A nutritional composition comprising digestible carbohydrates, protein and lipid, wherein the lipid is in the form of lipid globules which are at least partly coated on the surface with phospholipids, for improving the gut microbiota and reducing opportunistic pathogens in the gut of human infants.

### FIELD OF THE INVENTION

The invention relates to a nutritional composition for infants, in particular infant formula, follow-on formula or young child formula. The invention further relates to the improvement of gut microbiota in infants.

### BACKGROUND OF THE INVENTION

Human milk is the uncontested gold standard concerning infant nutrition. However, in some cases breastfeeding is inadequate or unsuccessful for medical reasons or not available because of a choice not to breastfeed. For such situations infant or follow-on formulas have been developed. Commercial infant formulas are commonly used today to provide supplemental or sole source of nutrition early in life. These formulas comprise a range of nutrients to meet the nutritional needs of the growing infant, and typically include fat, carbohydrate, protein, vitamins, minerals, and other nutrients helpful for optimal infant growth and development. Commercial infant formulas are designed to mimic, as closely as possible, the composition and function of human milk.

Human milk lipids are known to have a distinct physical structure composed of large lipid globules with a mode diameter, based on volume, of about 4 μm existing of a triglyceride core coated by a tri-layer of membranes, the milk fat globule membrane (MFGM). Standard infant formula's typically have lipid droplets with a mode diameter, based on volume, of about 0.3-0.5 μm due to industrial processing procedures applied to achieve stable products, and the lipid droplets are not surrounded by MFGM but mostly by milk proteins. Infant formula with lipid globules with an architecture more similar to the lipid globules in human milk have been described (e.g. WO2010/027258 or WO2010/027259).

US 2022/386675 A1 describes a method for non-therapeutic improvement of the postnatal growth trajectory or body development in a Caesarean born infant by administration of a nutritional composition selected from an infant formula and a follow on formula comprising carbohydrates, protein and lipid, wherein the lipid globules have such an architecture more similar to human milk lipid globules.

It is known in the art that some nutritional ingredients, besides prebiotic non-digestible oligosaccharides, beneficially affect gut microbiota. For example, it has been described that milk fat globule membrane (MFGM) may beneficially affect gut microbiota.

WO2009/082216 describes a composition comprising sphingophospholipid or its degradation product and at least one non-digestible carbohydrate for providing and/or maintaining an optimal intestinal microbiota.

Lopez et al., evaluated whether the specific composition and structure of the MFGM and milk polar lipid assemblies, associated with their nutritional and health benefits, could be used to tailor functional emulsions bioinspired by the MFGM-coated milk fat globules.

Berding et al. (2016), doi: 10.1097/MPG.0000000000001200, describes a study wherein two-day-old male piglets (n=24) were fed formula (CONT) or formula with polydextrose (1.2 g/100 g diet), galactooligosaccharides (3.5 g/100 g diet), bovine lactoferrin (0.3 g/100 g diet), and milk fat globule membrane-10 (2.5 g/100 g diet) (TEST) for 30 days. Microbial communities of TEST piglets differed from CONT in ascending colon (p=0.001) and feces (p=0.05).

He et al. (2019), doi: 10.1038/s41598-019-47953-4 studied the fecal microbiome and metabolome of infants fed a bovine MFGM supplemented experimental formula (EF) and compared to infants fed standard formula (SF) and a breast-fed reference group. The impact of MFGM on the fecal microbiome was moderate; the fecal metabolome of EF-fed infants showed a significant reduction of several metabolites including lactate, succinate, amino acids and their derivatives from that of infants fed SF.

Lee et al. (2020), doi: 10.1002/mnfr.202000603 describes a study wherein the serum metabolome and fecal microbiota are analyzed using 1H NMR spectroscopy and 16S rRNA gene sequencing respectively in a cohort of Chinese infants given a standard formula or a formula supplemented with an MFGM-enriched whey protein fraction. MFGM supplementation did not induce significant compositional changes in the fecal microbiota but suppressed microbial diversity and altered microbiota-associated metabolites.

Nevertheless, there is still a need in the art for nutritional compositions which reduce the chance for opportunistic pathogens, while promoting an increase in beneficial bacteria and thereby improving gut microbiota and (gut) health.

Tan et al. (2020), doi: 10.6084/m9.figshare.12689891, describes that gut microbiota modulation plays a role in the treatment and prevention of (gastro-intestinal) infections.

Melli et al. (2015), doi: 10.1016/j.aller.2015.01.013, describes the link between gut microbiota and the development of allergies.

Low et al. (2017), doi 10.3920/BM2017.0020, describes a link between an elevated Klebsiella/Bifidobacterium ratio in early infancy and development of paediatric allergy in childhood.

Di Costanzo et al. (2020), doi: :10.3390/ijms21155275, describes that gut dysbiosis early in life is connected to the development of food allergies.

Therefore, improving the gut microbiota of a human subject results in improvement of the subject's overall health, in particular in the treatment and/or prevention of gut dysbiosis, infections, and/or allergies

### SUMMARY OF THE INVENTION

The inventors of the present invention have surprisingly found that feeding an infant with a nutritional composition comprising large, phospholipid coated lipid globules improves the gut microbiota of said infant and brings it closer to the gut microbiota observed in breastfed infants. More in particular, the gut microbiota is improved by decreasing the relative abundance of opportunistic pathogens in the gut to the relative abundance levels of opportunistic pathogens observed in breastfed infants. The gut microbiota is also improved by increasing the relative abundance of beneficial bacteria and/or by reducing the ratio between the relative abundance of opportunistic bacteria and the relative abundance of beneficial bacteria.

It is considered beneficial to obtain a gut microbiota similar to the gut microbiota of breastfed infants. For infants in the first few months after birth it is particularly advantageous to have a reduction in opportunistic pathogens because the gut microbiota is strongly developing at this age. The presence of higher levels of opportunistic pathogens in early life has been associated with a compromised gut microbiota and this has been described as risk factor for childhood infections and non-communicable diseases (NCDs) such as allergy.

Without wishing to be bound to a theory, the inventors believe that decreasing the relative abundance of opportunistic pathogens, preferably Enterobacterales, while increasing the relative abundance of beneficial bacteria, preferably Bifidobacteriaceae, has the effect of reducing the risk of developing infections and allergy.

Hence, a first aspect of the invention pertains to a nutritional composition, selected from infant formula, follow-on formula and young child formula, comprising digestible carbohydrates, protein and lipid, wherein the lipid is in the form of lipid globules, wherein
a. said lipid globules have a volume-weighed mode diameter of at least 1.0 μm; and/or at least 45 vol. %, based on total lipid volume, of the lipid globules have a diameter between 2 and 12 μm; and
b. the lipid comprises 0.5 to 20 wt. % phospholipids based on total lipid and wherein the lipid globules are at least partly coated on the surface with a layer of phospholipids.
   for use in improving the gut microbiota in a human infant.

Without wishing to be bound by any theory, the presence of phospholipids, such MFGM, in the coating of the larger lipid globules in the nutritional composition, results in an improved beneficial effect on the gut microbiota compared to a nutritional composition wherein the phospholipids are not present in the coating of the lipid globules (either not present in the composition or separately present). It is hypothesized that due to the supramolecular lipid structure, both the phospholipid coating and the larger size of the lipid globules, a larger proportion of MFGM is capable of reaching the colon where the gut microbiota is located and therefor results in an improved effect on the gut microbiota.

### DETAILED DESCRIPTION OF THE INVENTION

The invention thus concerns a nutritional composition, selected from infant formula, follow-on formula and young child formula, comprising digestible carbohydrates, protein and lipid, wherein the lipid is in the form of lipid globules, wherein
a. said lipid globules have a volume-weighed mode diameter of at least 1.0 μm; and/or at least 45 vol. %, based on total lipid volume, of the lipid globules have a diameter between 2 and 12 μm; and
b. the lipid comprises 0.5 to 20 wt. % phospholipids based on total lipid and wherein the lipid globules are at least partly coated on the surface with a layer of phospholipids,
   for use in improving the gut microbiota in a human infant.

For some jurisdictions, the invention may also be worded as a method for improving the gut microbiota in a human infant, said method comprising the administration of a nutritional composition, selected from infant formula, follow-on formula and young child formula, comprising digestible carbohydrates, protein and lipid, wherein the lipid is in the form of lipid globules, wherein
a. said lipid globules have a volume-weighed mode diameter of at least 1.0 μm; and/or at least 45 vol. %, based on total lipid volume, of the lipid globules have a diameter between 2 and 12 μm; and
b. the lipid comprises 0.5 to 20 wt. % phospholipids based on total lipid and wherein the lipid globules are at least partly coated on the surface with a layer of phospholipids.

For some jurisdictions, the invention may also be worded as the use of digestible carbohydrates, protein and lipid in the manufacture of a nutritional composition for improving the gut microbiota in a human infant, wherein the nutritional composition is selected from infant formula, follow-on formula and young child formula, and wherein the lipid is in the form of lipid globules, wherein
a. the lipid globules have a mode diameter based on volume of at least 1 μm; and/or at least 45 vol. %, based on total lipid volume, of the lipid globules have a diameter between 2 and 12 μm; and
b. the lipid comprises 0.5 to 20 wt. % phospholipids based on total lipid and wherein the lipid globules are at least partly coated on the surface with a layer of phospholipids.

The invention can also be worded as the use of a nutritional composition, selected from infant formula, follow-on formula and young child formula, which comprises digestible carbohydrates, protein and lipid, wherein the lipid is in the form of lipid globules, wherein
a. the lipid globules have a mode diameter based on volume of at least 1 μm; and/or at least 45 vol. %, based on total lipid volume, of the lipid globules have a diameter between 2 and 12 μm; and
b. the lipid comprises 0.5 to 20 wt. % phospholipids based on total lipid and wherein the lipid globules are at least partly coated on the surface with a layer of phospholipids,
   for improving the gut microbiota in a human infant.

In some jurisdictions administering a nutritional composition to an infant is considered non-therapeutic. In those instances the invention may be worded as defined above by way of a method comprising administering a nutritional composition. For clarity, the method can also be defined as a non-therapeutic method. By definition, the words “non-therapeutic” exclude any therapeutic effect.

The term “gut microbiota” as used herein refers to all microorganisms, including bacteria, archaea, virus and fungi, that are found in the digestive tract of a human subject.

“Improving the gut microbiota in a human infant” as used herein, preferably refers to preventing or treating a compromised gut microbiota in a human infant or in other words, preventing or treating gut dysbiosis in a human infant.

The term “gut dysbiosis” as used herein refers to a disruption of the gut microbiome resulting in an imbalance in the gut microbiota.

Preferably, the improvement of gut microbiota is by decreasing the relative abundance of opportunistic pathogens in the gut microbiota and/or by increasing the relative abundance of beneficial bacteria in the gut microbiota and/or by decreasing the ratio between the relative abundance of opportunistic pathogens and the relative abundance of beneficial bacteria in the gut microbiota. More preferably, the improvement of gut microbiota is by decreasing the relative abundance of opportunistic pathogens in the gut microbiota.

Preferably, the opportunistic pathogens are selected from the phyla Proteobacteria and/or Bacillota. More preferably, the opportunistic pathogens are selected from Enterobacteriales and Clostridiaceae, Most preferably, the opportunistic pathogens are Enterobacterales.

Preferably, the Proteobacteria are Gammaproteobacteria, more preferably are Enterobacterales, even more preferably Enterobacteriaceae and yet even more preferably the Enterobacteriaceae are one or more of the genus Citrobacter, Enterobacter, Escherichia, Klebsiella, Proteus, Providencia, Salmonella, Shigella, and Yersinia. Most preferably the Enterobacteriaceae are one of more of the genus Enterobacter, Escherichia, Klebsiella and Shigella. Preferably, the Bacillota are Clostridiaceae.

Preferably the beneficial bacteria are selected from Lactobacillaceae and/or Bifidobacteriaceae, more preferably Bifidobacteriaceae.

Preferably, the improvement of gut microbiota is by reducing the relative abundance Enterobacterales in the gut microbiota and/or by increasing the relative abundance of Bifidobacteriaceae in the gut microbiota and/or by decreasing the ratio between the relative abundance of Enterobacterales and the relative abundance of Bifidobacteriaceae in the gut microbiota.

In a preferred embodiment, the decrease of the relative abundance of opportunistic pathogens in the human infant is compared to an human infant, whom consumed a nutritional composition selected from infant formula, follow-on formula and young child formula, comprising digestible carbohydrates, protein and lipid, wherein the lipid is in the form of lipid globules, wherein
a. the lipid globules have a volume-weighted mode diameter of 0.3-0.5 μm and less than 45 vol. %, based on total lipid volume, of the lipid globules have a diameter above 2 μm; and
b. the lipid comprises 0-20 wt. % phospholipids based on total lipid and wherein the lipid globules are not coated on the surface with phospholipids.

More preferably, the decrease of the relative abundance of opportunistic pathogens in the human infant is compared to an human infant, whom consumed a nutritional composition selected from infant formula, follow-on formula and young child formula, comprising digestible carbohydrates, protein and lipid, wherein the lipid is in the form of lipid globules, wherein
a. the lipid globules have a volume-weighted mode diameter of 0.3-0.5 μm and less than 45 vol. %, based on total lipid volume, of the lipid globules have a diameter above 2 μm; and
b. the lipid comprises 0.5 to 20 wt. % phospholipids based on total lipid and wherein the lipid globules are at least partly coated on the surface with a layer of phospholipids.

In a preferred embodiment, the increase of the relative abundance of beneficial bacteria in the human infant is compared to an human infant, whom consumed a nutritional composition selected from infant formula, follow-on formula and young child formula, comprising digestible carbohydrates, protein and lipid, wherein the lipid is in the form of lipid globules, wherein
a. the lipid globules have a volume-weighted mode diameter of 0.3-0.5 μm and less than 45 vol. %, based on total lipid volume, of the lipid globules have a diameter above 2 μm; and
b. the lipid comprises 0-20 wt. % phospholipids based on total lipid and wherein the lipid globules are not coated on the surface with phospholipids.

More preferably, the increase of the relative abundance of beneficial bacteria in the human infant is compared to an human infant, whom consumed a nutritional composition selected from infant formula, follow-on formula and young child formula, comprising digestible carbohydrates, protein and lipid, wherein the lipid is in the form of lipid globules, wherein
a. the lipid globules have a volume-weighted mode diameter of 0.3-0.5 μm and less than 45 vol. %, based on total lipid volume, of the lipid globules have a diameter above 2 μm; and
b. the lipid comprises 0.5 to 20 wt. % phospholipids based on total lipid and wherein the lipid globules are at least partly coated on the surface with a layer of phospholipids.

In a preferred embodiment, the decrease of the ratio between the relative abundance of opportunistic pathogens and the relative abundance of beneficial bacteria in the human infant is compared to an human infant, whom consumed a nutritional composition selected from infant formula, follow-on formula and young child formula, comprising digestible carbohydrates, protein and lipid, wherein the lipid is in the form of lipid globules, wherein
a. the lipid globules have a volume-weighted mode diameter of 0.3-0.5 μm and less than 45 vol. %, based on total lipid volume, of the lipid globules have a diameter above 2 μm; and
b. the lipid comprises 0-20 wt. % phospholipids based on total lipid and wherein the lipid globules are not coated on the surface with phospholipids.

More preferably, the decrease of the ratio between the relative abundance of opportunistic pathogens and the relative abundance of beneficial bacteria in the human infant is compared to an human infant, whom consumed a nutritional composition selected from infant formula, follow-on formula and young child formula, comprising digestible carbohydrates, protein and lipid, wherein the lipid is in the form of lipid globules, wherein
a. the lipid globules have a volume-weighted mode diameter of 0.3-0.5 μm and less than 45 vol. %, based on total lipid volume, of the lipid globules have a diameter above 2 μm; and
b. the lipid comprises 0.5 to 20 wt. % phospholipids based on total lipid and wherein the lipid globules are at least partly coated on the surface with a layer of phospholipids.

The use according to the invention preferably reduces the risk of developing infections in early life, preferably gut infections in early life. In another embodiment, the use according to the invention preferably reduces the risk of developing allergy, more preferably reduces the risk of developing food allergy.

In a preferred embodiment, the human infant is at risk of having a compromised gut microbiota. Preferably, the human infant at risk of having a compromised gut microbiota is selected from the group of infants born via caesarean section, preterm infants, infants born from an overweight or obese mother, infants born from a mother who received antibiotics, infants receiving or having received antibiotics, infants receiving or having received proton pump inhibitors, formula fed infants, or combinations thereof. More preferably, the human infant at risk of having a compromised gut microbiota is selected from the group of infants born via caesarean section, preterm infants and infants born from a mother who received intrapartum antibiotics. Most preferably, the human infant at risk of having a compromised gut microbiota is an infant born via caesarean section

“Infants born from a mother who received antibiotics” as used herein refers to infants born from a mother who received antibiotics in the two weeks preceding the delivery or during the delivery (intrapartum). Preferably, “infants born from a mother who received antibiotics” refers to infants born from a mother who received intrapartum antibiotics.

Preferably, the human infant is aged 0-36 months, more preferably 0-24 months, even more preferably 0-12 months and most preferably 0-6 months. The term “early life” as used herein refers to the first 0-36 months of life, preferably to the first 0-24 months of life, more preferably to the first 0-12 months and most preferably to the first 0-6 months of life.

### Lipid Globule Size

The lipid is present in the nutritional composition in the form of lipid globules. When the nutritional composition is in liquid form, these lipid globules are emulsified in the aqueous phase. Alternatively, when the nutritional composition is in powder form, the lipid globules are present in the powder and the powder is suitable for reconstitution with water or another food grade aqueous phase. The lipid globules comprise a core and a surface.

The lipid globules in the nutritional composition preferably have mode diameter, based on volume, of at least 1.0 μm, more preferably at least 3.0 μm, and most preferably at least 4.0 μm. Preferably, the lipid globules have a mode diameter, based on volume, between 1.0 and 10 μm, more preferably between 2.0 and 8.0 μm, even more preferably between 3.0 and 7.0 μm, and most preferably between 4.0 μm and 6.0 μm.

Alternatively, or preferably in addition, the size distribution of the lipid globules is preferably in such a way that at least 45 volume % (vol. %), preferably at least 55 vol. %, even more preferably at least 65 vol. %, and most preferably at least 75 vol. % of the lipid globules have a diameter between 2 and 12 μm. In a preferred embodiment, at least 45 vol. %, preferably at least 55 vol. %, more preferably at least 65 vol. %, and most preferably at least 75 vol. % of the lipid globules have a diameter between 2 and 10 μm. In a more preferred embodiment, at least 45 vol. %, more preferably at least 55 vol. %, yet even more preferably at least 65 vol. %, and most preferably at least 75 vol. % of the lipid globules have a diameter between 4 and 10 μm. Preferably less than 5 vol. % of the lipid globules have a diameter above 12 μm.

The percentage of lipid globules is based on volume of total lipid. The mode diameter relates to the diameter which is the most present based on volume of total lipid, or the peak value in a graphic representation, having on the X-axis the diameter and on the Y-axis the volume (%).

The volume of the lipid globules and its size distribution can suitably be determined using a particle size analyzer such as a Mastersizer 2000 (Malvern Instruments, Malvern, UK), for example by the method described in Michalski et al, 2001, Lait 81: 787-796.

### Phospholipid

The lipid in the nutritional composition comprises 0.5 to 20 wt. % phospholipids based on total lipid and wherein the lipid globules are at least partly coated on the surface with a layer of phospholipids. Preferably the nutritional composition comprises 0.6 to 10 wt. %, more preferably 0.7 to 8 wt. %, even more preferably 0.8 to 8 wt. % even more preferably 1 to 5 wt. % phospholipid based on total lipid.

Phospholipids are amphipathic of nature and include glycerophospholipids and sphingomyelin. By ‘coating’ is meant that the outer surface layer of the lipid globules comprises phospholipid, whereas phospholipid is virtually absent in the core of the lipid globule. A suitable way to determine whether phospholipid is located on the surface of lipid globules is confocal laser scanning microscopy or transmission electron microscopy; see for instance Gallier et al. (A novel infant milk formula concept: Mimicking the human milk fat globule structure, Colloids and Surfaces B: Biointerfaces 136 (2015) 329-339).

The nutritional composition preferably comprises glycerophospholipids. Examples of glycerophospholipids are phosphatidylcholine (PC), phosphatidylserine (PS), phosphatidylethanolamine (PE), phosphatidylinositol (PI) and phosphatidylglycerol (PG). Preferably the nutritional composition comprises one or more of PC, PS, PI and PE, more preferably the nutritional composition comprises at least PC.

The nutritional composition preferably comprises sphingomyelin. Sphingomyelins have a phosphorylcholine or phosphorylethanolamine molecule esterified to the 1-hydroxy group of a ceramide. They are classified as phospholipid as well as sphingolipid, but are not classified as a glycerophospholipid nor as a glycosphingolipid. Preferably the nutritional composition comprises 0.05 to 10 wt. % sphingomyelin based on total lipid, more preferably 0.1 to 5 wt. %, even more preferably 0.2 to 2 wt. %. Preferably the nutritional composition comprises at least 5 wt. %, more preferably 5 to 40 wt. % sphingomyelin based on total phospholipid, more preferably 10 to 35 wt. %, even more preferably 15 to 35 wt. % sphingomyelin, based on total phospholipid.

The nutritional composition preferably comprises glycosphingolipids. The term glycosphingolipids in the present context particularly refers to glycolipids with an amino alcohol sphingosine. The sphingosine backbone is O-linked to a charged head-group such as ethanolamine, serine or choline backbone. The backbone is also amide linked to a fatty acyl group. Glycosphingolipids are ceramides with one or more sugar residues joined in a beta-glycosidic linkage at the 1-hydroxyl position, and include gangliosides. Preferably the nutritional composition contains gangliosides, more preferably at least one ganglioside selected from the group consisting of GM3 and GD3. Preferably the nutritional composition comprises 0.1 to 10 wt. % glycosphingolipids based on total lipid, more preferably 0.5 to 5 wt. %, even more preferably 2 to 4 wt. % glycosphingolipids, based on total lipid.

The nutritional composition preferably comprises cholesterol. The nutritional composition preferably comprises at least 0.005 wt. % cholesterol based on total lipid, more preferably at least 0.02 wt. %, more preferably at least 0.05 wt. %, even more preferably at least 0.1 wt. % cholesterol based on total lipid. Preferably the amount of cholesterol does not exceed 10 wt. % based on total lipid, more preferably does not exceed 5 wt. %, even more preferably does not exceed 1 wt. % of cholesterol based on total lipid in the nutritional composition.

Preferred sources for providing the phospholipid, glycosphingolipid and/or cholesterol are egg lipids, milk fat, buttermilk fat and butter serum fat (such as beta serum fat). Another preferred source for phospholipid, particularly PC, is soy lecithin and/or sunflower lecithin.

The nutritional composition preferably comprises phospholipid derived from mammalian milk. Preferably the nutritional composition comprises phospholipid and glycosphingolipid derived from mammalian milk. Preferably also cholesterol is obtained from mammalian milk. The nutritional composition preferably comprises phospholipid, glycosphingolipid and/or cholesterol from mammalian milk of cows, mares, sheep, goats, buffalos, horses and camels. More preferably the nutritional composition comprises phospholipid, glycosphingolipid and/or cholesterol from cow's milk.

Phospholipid derived from mammalian milk includes preferably phospholipid that is isolated from milk lipid, cream lipid, cream serum lipid, butter serum lipid (beta serum lipid), whey lipid, cheese lipid and/or buttermilk lipid. Buttermilk lipid is typically obtained during the manufacture of buttermilk. Butter serum lipid or beta serum lipid is typically obtained during the manufacture of anhydrous milk fat from butter. Preferably the phospholipid, glycosphingolipid and/or cholesterol is obtained from milk cream. Suitable commercially available sources for phospholipid from milk are BAEF, SM2, SM3 and SM4 powder of Corman, Salibra of Glanbia, Vivinal MFGM of FrieslandCampina and LacProdan MFGM-10 or PL20 from Arla.

The use of phospholipid from mammalian milk fat advantageously comprises the use of milk fat globule membranes, which are more reminiscent to the situation in human milk. The concomitant use of phospholipid derived from mammalian milk and triglycerides derived from vegetable lipids therefore enables the manufacture of coated lipid globules with a coating more similar to human milk, while at the same time providing an optimal fatty acid profile.

Preferably the phospholipid is derived from mammalian milk lipid, more preferably from milk fat globule membrane (MFGM). Preferably the phospholipid is derived from cow's milk lipid, more preferably from cow's MFGM.

Preferably the nutritional composition comprises phospholipid and glycosphingolipid and more preferably the weight ratio of phospholipid:glycosphingolipid is from 2:1 to 12:1, more preferably from 2:1 to 10:1 and even more preferably 2:1 to 5:1.

Methods for obtaining lipid globules with an increased size and/or coating with phospholipid are for example described in WO 2010/0027258 and WO 2010/0027259.

### Lipid

The nutritional composition according to the present use comprises lipid. Lipid in the present invention comprises one or more selected from the group consisting of triglycerides, polar lipids (such as phospholipids, cholesterol, glycolipids, sphingomyelin), free fatty acids, monoglycerides and diglycerides.

The lipids provide preferably 30 to 60% of the total calories of the nutritional composition. More preferably the nutritional composition comprises lipid providing 35 to 55% of the total calories, even more preferably the nutritional composition comprises lipids providing 40 to 50% of the total calories. The lipids are preferably present in an amount of 4 to 6 g per 100 kcal. When in liquid form, e.g. as a ready-to-feed liquid, the nutritional composition preferably comprises 2.1 to 6.5 g lipids per 100 ml, more preferably 3.0 to 4.0 g per 100 ml. Based on dry weight the nutritional composition preferably comprises 10 to 50 wt. %, more preferably 12.5 to 40 wt. % lipids, even more preferably 19 to 30 wt. % lipids.

The lipid preferably comprises vegetable lipids. The presence of vegetable lipids advantageously enables an optimal fatty acid profile, high in polyunsaturated fatty acids and/or more reminiscent to human milk fat. Lipids from mammalian milk alone, e.g. cow's milk, do not provide an optimal fatty acid profile. The amount of essential fatty acids is too low in mammalian milk.

Preferably the nutritional composition comprises at least one, preferably at least two vegetable lipid sources selected from the group consisting of linseed oil (flaxseed oil), rape seed oil (such as colza oil, low erucic acid rape seed oil and canola oil), sunflower oil, high oleic sunflower oil, safflower oil, high oleic safflower oil, olive oil, coconut oil, palm oil and palm kernel oil.

In one preferred embodiment, the nutritional composition comprises 5 to 100 wt. % vegetable lipids based on total lipids, more preferably 10 to 95 wt. %, more preferably 20 to 80 wt. %, even more preferably 25 to 75 wt. %, most preferably 40 to 60 wt. %. It is noted therefore that the nutritional composition also may comprise non-vegetable lipids. Non-vegetable lipids may include mammalian milk fat, mammalian milk derived lipid as a preferred source of phospholipid, and fish, marine and/or microbial oils as source of LC-PUFA.

### Palmitic Acid (PA) at Sn-2 Position of Triglyceride

Triglycerides are preferably the major fraction of the lipids in the nutritional composition. Triglycerides comprise a glycerol moiety to which, via ester bonds, three fatty acid residues are attached, which may be the same or different, and which are generally chosen from saturated and unsaturated fatty acids containing 4 to 26 carbon atoms. Such triglycerides may differ in the fatty acid residues that are present and/or may differ in the respective position(s) of the fatty acid residues to the glycerol backbone (e.g. in the sn-1, sn-2 and/or sn-3 position).

Preferably the nutritional composition comprises at least 70 wt. %, more preferably at least 80 wt. %, more preferably at least 85 wt. % triglycerides based on total lipids, even more preferably at least 90 wt. % triglycerides based on total lipids, even more preferably at least 95 wt. % triglycerides based on total lipids.

Further reduction of the relative abundance of opportunistic pathogens and thereby improvement of the gut microbiota in human infants was observed when the lipid component had an increased amount of palmitic acid (PA) acid located at the sn-2 position in a triglyceride, based on total PA. PA relates to palmitic acid and/or acyl chains (C16:0).

Lipids that can be used to enhance the amount of PA located at the sn-2 position in triglycerides based on total PA are commercially available—e.g. from Loders Croklaan under the name Betapol™ and/or can be prepared in a manner known per se, for instance as described in EP 0698078 and/or EP 0758846. Another suitable source is InFat™ of Enzymotec. In case these lipids are obtained by trans-or interesterification of vegetable triglycerides, these sources are in the context of the present invention regarded as vegetable lipids.

A preferred source for triglycerides to enhance PA at the sn-2 or beta position in a triglyceride is mammalian milk fat, preferably non-human mammalian milk fat, more preferably cow's milk fat. Preferably mammalian milk fat, in particular cow's milk fat, is used in the form of anhydrous milk fat, butter oil, butter fat or butter. Preferably, the source of the mammalian milk fat is in a homogenous fat phase, such as butter oil or anhydrous milk fat, and not in the form of oil in water emulsion such as cream, since the lipid globules of the present invention can be more easily prepared during the manufacture of the nutritional composition for the use according to the present invention, when the lipid is added to the aqueous phase as homogenous fat phase, upon which the mixture is treated to form an emulsion.

Preferably the amount of the source of lipid, comprising triglyceride with an increased amount of palmitic acid residues in the sn-2 position of a triglyceride, that is comprised in the lipid of the nutritional composition, is between 10 and 99.5 wt. %, more preferably between 15 and 85 wt. % based on total lipid, more preferably between 20 and 75 wt. %, more preferably between 25 and 65 wt. %, even more preferably between 30 and 60 wt. % based on total lipid. Preferably the nutritional composition comprises mammalian milk fat between 5 and 95 wt. %, more preferably between 20 and 80 wt. % based on total lipid, more preferably between 25 and 75 wt. %, even more preferably between 40 and 60 wt. % based on total lipid.

In a particularly preferred embodiment, the lipid in the nutritional composition comprises:
a. 30 to 90 wt. % vegetable fat based on total lipid, and
b. 10 to 70 wt. % mammalian milk fat based on total lipid.

More preferably, the lipid in the nutritional composition comprises:
a. 35 to 75 wt. % vegetable fat based on total lipid, and
b. 25 to 65 wt. % mammalian milk fat based on total lipid.

Most preferably, the lipid in the nutritional composition comprises:
a. 40 to 60 wt. % vegetable fat based on total lipid, and
b. 40 to 60 wt. % mammalian milk fat based on total lipid.

The sources of the lipid in the nutritional composition are preferably chosen such that the amount of palmitic acid (PA) that is present in the total lipid of the nutritional composition is at least 10 wt. % based on total fatty acids, preferably at least 15 wt. %. Preferably the amount of PA that is present in the total lipid is below 30 wt. % based on total fatty acids. More preferably the amount of PA that is present in the lipid is from 15 to 24 wt. % based on total fatty acids, even more preferably from 15 to 19 wt. %, even more preferably from 16 to 19 wt. %.

The lipids in the nutritional composition are preferably chosen such that, based on the total PA, at least 15 wt. %, preferably at least 20 wt. %, more preferably at least 25 wt. %, more preferably at least 30 wt. % PA is in the sn-2 or beta position in a triglyceride. Preferably the amount of PA in the sn-2 position in a triglyceride is not more than 45 wt. %, preferably not more than 40 wt. % based on total PA. Preferably the amount of PA in the sn-2 position in a triglyceride is from 25 to 40 wt. % based on total PA present in the total lipid.

In the context of the present invention, a weight percentage of fatty acids based on total fatty acids is calculated as if all fatty acids are free fatty acids, hence it is not taken into account whether a fatty acid is attached to a glycerol backbone or not.

### Fatty Acid Composition

SFA relates to saturated fatty acids and/or acyl chains, MUFA relates to mono-unsaturated fatty acid and/or acyl chains, PUFA refers to polyunsaturated fatty acids and/or acyl chains with 2 or more unsaturated bonds; LC-PUFA refers to long chain polyunsaturated fatty acids and/or acyl chains comprising at least 20 carbon atoms in the fatty acyl chain and with 2 or more unsaturated bonds; DHA refers to docosahexaenoic acid and/or acyl chain (22:6, n3); EPA refers to eicosapentaenoic acid and/or acyl chain (20:5 n3); ARA refers to arachidonic acid and/or acyl chain (20:4 n6); DPA refers to docosapentaenoic acid and/or acyl chain (22:5 n3). n3 or omega 3 PUFA refers to polyunsaturated fatty acids and/or acyl chains with 2 or more unsaturated bonds and with an unsaturated bond at the third carbon atom from the methyl end of the fatty acyl chain, n6 or omega 6 PUFA refers to polyunsaturated fatty acids and/or acyl chains with 2 or more unsaturated bonds and with an unsaturated bond at the sixth carbon atom from the methyl end of the fatty acyl chain.

The nutritional composition according to the present use preferably comprises LA, which refers to linoleic acid and/or acyl chain (18:2 n6). LA is an n6 PUFA and the precursor of n6 LC-PUFA and is an essential fatty acid as it cannot be synthesized by the human body. LA preferably is present in a sufficient amount in order to promote a healthy growth and development, yet in an amount as low as possible to prevent negative, competitive, effects on the formation of n3 PUFA and a too high n6/n3 ratio. The nutritional composition therefore preferably comprises less than 25 wt. %, more preferably less than 20 wt. %, more preferably less than 15 wt. % LA based on total fatty acids. The nutritional composition preferably comprises at least 5 wt. % LA based on fatty acids, preferably at least 7.5 wt. %, more preferably at least 10 wt. % based on total fatty acids.

The nutritional composition preferably comprises ALA, which refers to alpha-linolenic acid and/or acyl chain (18:3 n3). ALA is a n3 PUFA and the precursor of n3 LC-PUFA and is an essential fatty acid as it cannot be synthesized by the human body. Preferably ALA is present in a sufficient amount to promote a healthy growth and development of the infant. The nutritional composition therefore preferably comprises at least 0.5 wt. %, more preferably at least 1.0 wt. %, more preferably the nutritional composition comprises at least 1.5 wt. %, even more preferably at least 2.0 wt. % ALA based on total fatty acids. Preferably the nutritional composition comprises less than 10 wt. % ALA, more preferably less than 5.0 wt. % ALA based on total fatty acids.

The weight ratio LA/ALA preferably is well balanced in order to ensure an optimal n6/n3 PUFA, n6/n3 LC PUFA and DHA/ARA ratio in the cellular membranes. Therefore, the nutritional composition preferably comprises a weight ratio of LA/ALA from 2 to 20, more preferably from 3 to 15, more preferably from 5 to 12, more preferably from 5 to 10. Preferably the n6 PUFA/n3 PUFA weight ratio is from 3 to 20, more preferably from 3 to 15, more preferably from 5 to 12, more preferably from 5 to 10.

Preferably, the nutritional composition comprises n3 LC-PUFA, such as EPA, DPA and/or DHA, more preferably DHA. As the conversion of ALA to DHA may be less efficient in infants, preferably both ALA and DHA are present in the nutritional composition. Preferably the nutritional composition comprises at least 0.05 wt. %, preferably at least 0.1 wt. %, more preferably at least 0.2 wt. %, of DHA based on total fatty acids. Preferably the nutritional composition comprises not more than 2.0, preferably not more than 1.0 wt. %, of DHA based on total fatty acids.

The nutritional composition preferably comprises ARA. Preferably the nutritional composition comprises at least 0.05 wt. %, preferably at least 0.1 wt. %, more preferably at least 0.2 wt. %, of ARA based on total fatty acids. As the group of n6 fatty acids, especially ARA counteracts the group of n3 fatty acids, especially DHA, the nutritional composition preferably comprises relatively low amounts of ARA. Preferably the nutritional composition comprises not more than 2.0 wt. %, preferably not more than 1.0 wt. %, of ARA based on total fatty acids. Preferably the weight ratio between DHA and ARA is between % to 4/1, more preferably between ½ to 2/1, more preferably between 0.6 and 1.5.

### Digestible Carbohydrates

The nutritional composition comprises digestible carbohydrates. The digestible carbohydrates preferably provide 30 to 80% of the total calories of the nutritional composition. Preferably the digestible carbohydrates provide 40 to 60% of the total calories. Based on calories the nutritional composition preferably comprises of 5 to 20 g of digestible carbohydrates per 100 kcal, more preferably 7.5 to 15 g. When in liquid form, e.g. as a ready-to-feed liquid, the nutritional composition preferably comprises 3 to 30 g digestible carbohydrate per 100 ml, more preferably 6 to 20, even more preferably 7 to 10 g per 100 ml. Based on dry weight the nutritional composition preferably comprises 20 to 80 wt. %, more preferably 40 to 65 wt. % digestible carbohydrates.

Preferred digestible carbohydrate sources are lactose, glucose, sucrose, fructose, galactose, maltose, starch and maltodextrin. Lactose is the main digestible carbohydrate present in human milk. Lactose advantageously has a low glycemic index. The nutritional composition preferably comprises lactose. The nutritional composition preferably comprises digestible carbohydrate, wherein at least 35 wt. %, more preferably at least 50 wt. %, more preferably at least 75 wt. %, even more preferably at least 90 wt. %, most preferably at least 95 wt. % of the digestible carbohydrate is lactose. Based on dry weight the nutritional composition preferably comprises at least 25 wt. % lactose, preferably at least 40 wt. %.

### Protein

The nutritional composition comprises protein. The protein preferably provides 5 to 15% of the total calories. Preferably the nutritional composition comprises protein that provides 6 to 12% of the total calories. More preferably protein is present in the nutritional composition below 3.5 gram per 100 kcal, more preferably the nutritional composition comprises between 1.8 and 2.1 g protein per 100 kcal, even more preferably between 1.85 and 2.0 g protein per 100 kcal. The protein concentration in a nutritional composition is determined by the sum of protein, peptides and free amino acids. Based on dry weight the nutritional composition preferably comprises less than 12 wt. % protein, more preferably between 9.6 and 12 wt. %, even more preferably between 10 and 11 wt. %. Based on a ready-to-drink liquid product the nutritional composition preferably comprises less than 1.5 g protein per 100 ml, more preferably between 1.2 and 1.5 g, even more preferably between 1.25 and 1.35 g.

The source of the protein should be selected in such a way that the minimum requirements for essential amino acid content are met and satisfactory growth is ensured. Hence protein sources based on cows' milk proteins such as whey, casein and mixtures thereof and proteins based on soy, potato or pea are preferred. In case whey proteins are used, the protein source is preferably based on acid whey or sweet whey, whey protein isolate or mixtures thereof. Preferably the nutritional composition comprises at least 3 wt. % casein based on dry weight. Preferably the casein is intact and/or non-hydrolyzed.

### Non Digestible Carbohydrates

In one embodiment the nutritional composition preferably comprises non-digestible oligosaccharides. Preferably the nutritional composition comprises non-digestible oligosaccharides with a degree of polymerization (DP) between 2 and 250, more preferably 3 and 60. The non-digestible oligosaccharides advantageously further reduce of the relative abundance of opportunistic pathogens and improves the gut microbiota in human infants

Preferably the nutritional composition comprises fructo-oligosaccharides, galacto-oligosaccharides and/or galacturonic acid oligosaccharides, more preferably fructo-oligosaccharides and/or galacto-oligosaccharides, even more preferably galacto-oligosaccharides, most preferably transgalacto-oligosaccharides. In a preferred embodiment the nutritional composition comprises a mixture of galacto-oligosaccharides and fructo-oligosaccharides, more preferably transgalacto-oligosaccharides and fructo-oligosaccharides. Suitable non-digestible oligosaccharides are for example Vivinal®GOS (FrieslandCampina DOMO), Raftilin®HP or Raftilose® (Orafti).

Preferably, the nutritional composition comprises 80 mg to 2 g non-digestible oligosaccharides per 100 ml, more preferably 150 mg to 1.5 g, even more preferably 300 mg to 1 g per 100 ml. Based on dry weight, the nutritional composition preferably comprises 0.25 wt. % to 20 wt. %, more preferably 0.5 wt. % to 10 wt. %, even more preferably 1.5 wt. % to 7.5 wt. % of non-digestible oligosaccharides.

### Formula

The use according to the present invention requires the administration of an infant formula, a follow-on formula or a young child formula. This means that the composition that is administered is not human milk. It also means that the composition that is administered is not native cow's milk or native milk from another mammal. Alternatively, the terms as used herein, “infant formula” or “follow-on formula” or “young child formula” means that it concerns a composition that is artificially made or that it is synthetic. Hence in one embodiment the nutritional composition that is administered is an artificial infant formula or an artificial follow-on formula or an artificial young child formula or a synthetic infant formula or a synthetic follow-on formula or a synthetic young child formula.

In the present context, infant formula refers to nutritional compositions, artificially made, intended for infants of 0 to about 4 to 6 months of age and are intended as a substitute for human milk. Typically infant formulae are suitable to be used as sole source of nutrition. Such formulae are also known as starter formula. Formula for infants starting with at 4 to 6 months of life to 12 months of life are intended to be supplementary feedings to infants that start weaning on other foods. Such formulae are also known as follow-on formulae. Infant formulae and follow-on formulae are subject to strict regulations, for example for the EU Commission Directive 2006/141/EC. In the present context, young child formulae refers to nutritional compositions, artificially made, intended for infants of 12 months to 36 months, which are intended to be supplementary feedings to infants. Such formulae are also known as growing-up milks.

The nutritional composition is preferably an infant formula or a follow-on formula. More preferably the nutritional composition is an infant formula.

The nutritional composition is preferably an infant formula or follow-on formula and preferably comprises 3 to 7 g lipid/100 kcal, preferably 4 to 6 g lipid/100 kcal, more preferably 4.5 to 5.5 g lipid/100 kcal, preferably comprises 1.7 to 5 g protein/100 kcal, preferably 1.8 to 3.5 g protein/100 kcal, more preferably 1.8 to 2.1 g protein/100 kcal, more preferably 1.8 to 2.0 g protein/100 kcal and preferably comprises 5 to 20 g digestible carbohydrate/100 kcal, preferably 6 to 16 g digestible carbohydrate/100 kcal, more preferably 10 to 15 g digestible carbohydrate/100 kcal.

Preferably the nutritional composition is an infant formula or follow-on formula, when in a ready-to-drink format has an energy density of 60 kcal to 75 kcal/100 ml, more preferably 60 to 70 kcal/100 ml. This density ensures an optimal balance between hydration and caloric intake.

In one embodiment, the nutritional composition is a powder. Suitably, the nutritional composition is in a powdered form, which can be reconstituted with water or other food grade aqueous liquid, to form a ready-to drink liquid, or is in a liquid concentrate form that should be diluted with water to a ready-to-drink liquid. It was found that lipid globules maintained their size and coating when reconstituted.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### DESCRIPTION OF FIGURES

FIGS. 1a-c shows the relative abundance of three genera in the family of Enterobacteriaceae in the fecal microbiota for the Breastfed reference group, CONTROL group and the TEST group.

FIG. 2 shows the relative abundance of Clostridiaceae in the fecal microbiota for the Breastfed reference group, CONTROL group and the TEST group.

FIGS. 3a-b show the relative abundance of Enterobacterales and Bifidobacteriaceae, respectively, in the fecal microbiota of the fecal slurry fermented with IMF-A or IMF-1.

FIG. 4 shows the ratio between the relative abundance of Enterobacterales over Bifidobacteriaceae in the fecal microbiota of the fecal slurry fermented with IMF-A or IMF-1.

### EXAMPLES

### Example 1

A clinical study was conducted in 17 study centers in four countries including The Netherlands, Belgium, France and Singapore.

### Subjects and Study Design

Healthy term infants, with a gestational age between 37 weeks and 42 weeks, postnatal age ≤35 days, and either fully formula-fed or fully breastfed were eligible for participation. The study was designed as a randomized, double-blind, controlled, prospective, multi-country, equivalence trial. After enrolment, formula-fed infants (n=223) were randomly assigned to receive the TEST formula (n=115) or the CONTROL formula (n=108) ad libitum using region (Europe/Asia), sex (male/female) and infants' age at randomization (≤14 days/>14 days) as strata. Breastfed infants (n=88) served as a Reference group and were eligible if the mother intended to breastfeed exclusively for at least 13 weeks. During the study, infants were fully formula-fed or fully breast-fed. The groups of infants included both vaginally born infants and infants born by caesarean section

### Study Infant Formulas

The two study infant formulas used in this study were complete standard cow's milk-based infant formulas that comprised per 100 ml reconstituted formula 66 kcal, 1.3 g protein (intact protein with a casein/whey ratio of 40/60), 7.3 g digestible carbohydrates (mainly lactose), 3.4 g fat and 0.8 g short chain galacto-oligosaccharides (source Vivinal® GOS) and long chain fructo-oligosaccharides (source Raftilin HP®) in a 9/1 w/w ratio, and minerals, vitamins trace elements and other micronutrients as known in the art and in compliance with directives for infant formula. The formula was provided as a powder with the instruction to reconstitute with water. About 13.6 g powder was to be reconstituted to 100 ml water to obtain the reconstituted infant formula. The two study formulas only differed in lipid sources used and in lipid globule size (Table 1). The fatty acid composition was very similar between the CONTROL formula and the TEST formula, in saturated, mono-unsaturated and poly-unsaturated fatty acids, and in n3- and n6-PUFA content.

### Control Formula

The fat component comprised mainly vegetable fat (blend of palm oil, low erucic acid rape seed oil, coconut oil, high oleic sunflower oil, sunflower oil) and about 1.5 wt. % of an LC-PUFA premix (fish oil and microbial oil). No mammalian milk derived phospholipid was added.

### Test Formula

The fat component comprised of about 50 wt. % vegetable fat (blend of low erucic acid rape seed oil, coconut oil, high oleic sunflower oil, sunflower oil), about 44 wt. % bovine anhydrous milk fat, 1.5 wt. % LC-PUFA containing oil (fish oil and microbial oil), about 3.6 wt. % mammalian milk fat derived from buttermilk rich in milk phospholipid or milk fat globule membranes (milk phospholipid is about 1.5 wt. % based on total lipid). The lipid droplets in the TEST formula had a volume-based mode diameter of 5.6 μm and an interface predominantly composed of milk phospholipids following a production process as described in WO 2013/135739.

TABLE 1
Composition of the study formulas (per 100 ml)

| | CONTROL formula | | TEST formula | |
|---|---|---|---|---|
| Per 100 ml | Weight | | Weight | |
| Total lipid | 3.4 | g | 3.4 | g |
| Linoleic acid | 447 | mg | 447 | mg |
| Alpha linolenic acid | 82 | mg | 83 | mg |
| Arachidonic acid | 11 | mg | 12 | mg |
| Docosahexaenoic acid | 6.4 | mg | 6.6 | mg |
| Palmitic acid | 580 | mg | 566 | mg |
| sn-2 palmitic acid | 67 | mg | 202 | mg |
| Milk phospholipids | — | | 55 | mg |
| Volume-based mode | 0.5 | μm | 5.6 | μm |
| diameter lipid globules | | | | |

| | | |
|---|---|---|
| Volume % of lipid | <10 vol. % | >45 vol. % |
| globules with a diameter | | |
| between 2 and 12 μm | | |
| Coated with phospholipids | No | Yes |

Stool samples were obtained at 3 months of age during the intervention period and the fecal microbiota was determined by 16S rRNA sequencing. DNA was extracted from the stool samples as previously described (Wopereis, H. et al. Intestinal microbiota in infants at high risk for allergy: Effects of prebiotics and role in eczema development. J. Allergy Clin. Immunol. (2017)).

Primers Bact-0341F and Bact-0785R were used to amplify the region V3-V5 of the 16S rRNA gene (Klindworth, A. et al. Evaluation of general 16S ribosomal RNA gene PCR primers for classical and next-generation sequencing-based diversity studies. Nucleic Acids Res. 41, e1-e1 (2013)). Amplicons were sequenced on an Illumina MiSeq instrument (Illumina, San Diego, CA, USA) as previously described (Caporaso, J. G. et al. Ultra-high-throughput microbial community analysis on the Illumina HiSeq and MiSeq platforms. ISME J. 2012 68 6,1621-1624 (2012)). QIIME 1.9.0 was used to generate the OTUs table from the sequencing data (Caporaso, J. G. et al. QIIME allows analysis of high-throughput community sequencing data. Nat. Methods 7, 335-336 (2010)). Quality controls filter were applied as previously described (van den Elsen, L. W. J. et al. Prebiotic oligosaccharides in early life alter gut microbiome development in male mice while supporting influenza vaccination responses. Benef. Microbes 10, 279-291 (2019)). De novo OTU picking was performed with the USEARCH algorithm using a 97% sequence identity (Edgar, R. C. Search and clustering orders of magnitude faster than BLAST. Bioinformatics 26, 2460-2461 (2010)). The SILVA database (release version 1.1.9) was used for taxonomy assignment (Quast, C. et al. The SILVA ribosomal RNA gene database project: improved data processing and web-based tools. Nucleic Acids Res. 41, D590-D596 (2013)). Singletons and low abundant OTUs with a relative abundance <0.002% were excluded for downstream analysis.

### Results

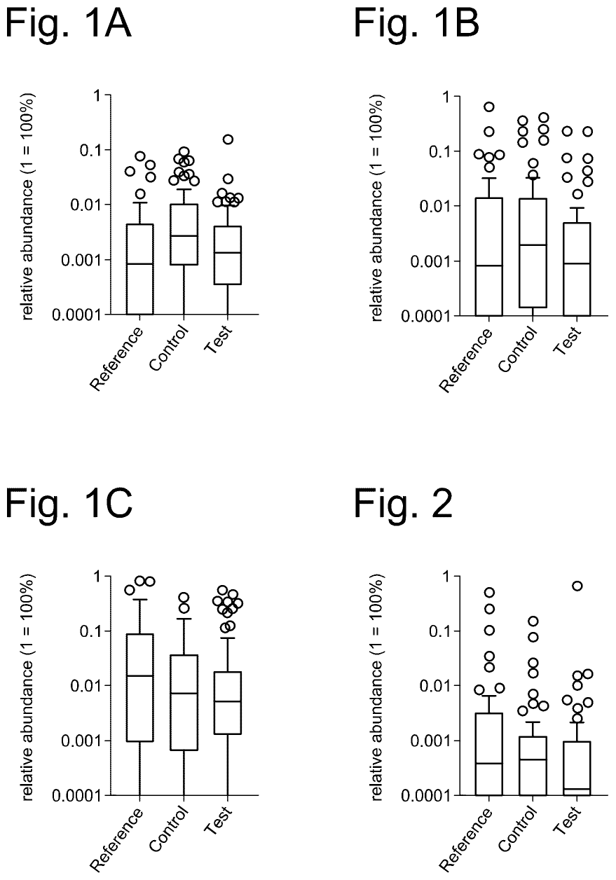

FIGS. 1a-c shows the relative abundance of three genera in the family of Enterobacteriaceae in the fecal microbiota for the Breastfed reference group, CONTROL group and the TEST group.

FIG. 2 shows the relative abundance of the family of Clostridiaceae in the fecal microbiota for the Breastfed reference group, CONTROL group and the TEST group.

The genera Escherichia, Shigella, Enterobacter, Klebsiella, and the family of Clostridiaceae are known to encompass opportunistic pathogens. In the figures it may be observed that in the TEST group the relative abundance of these microorganisms is lower compared to the CONTROL group and is more similar to the relative abundance levels observed in the Breastfed reference group.

### Example 2

An in vitro study was conducted to compare the effect of an infant formula according to the invention having large lipid globules (IMF-1) with a similar infant formula having smaller lipid globules (IMF-A).

### Study Infant Formulas

The two study infant formulas used in this example were complete standard cow's milk-based infant formulas having the same composition, except for the size of the lipid globules. The infant formulas comprised per 100 ml reconstituted formula 66 kcal, 1.3 g protein (intact protein with a casein/whey ratio of 40/60), 7.3 g digestible carbohydrates (mainly lactose), 3.4 g fat and 0.8 g short chain galacto-oligosaccharides (source Vivinal® GOS) and long chain fructo-oligosaccharides (source Raftilin HP®) in a 9/1 w/w ratio, and minerals, vitamins trace elements and other micronutrients as known in the art and in compliance with directives for infant formula.

The lipid source was the same for both study formulas. The fat component comprised vegetable fat (blend of low erucic acid rape seed oil, coconut oil, high oleic sunflower oil, sunflower oil), bovine anhydrous milk fat, LC-PUFA containing oil (fish oil and microbial oil). In addition, a whey protein concentrate enriched in MFGM also provided additional milk fat including phospholipids (milk phospholipid was about 1.5 wt. % based on total lipid). The resulting fatty acid composition was identical for both study formulas, in terms of saturated, mono-unsaturated and poly-unsaturated fatty acids, and in n3- and n6-PUFA content. The two study formulas only differed in lipid globule size (Table 2).

The lipid globules of both IMF-A and IMF-1 had a coating comprising phospholipids. IMF-A was prepared by a standard production process, wherein the milk-derived phospholipids were added before homogenization. IMF-1 was prepared following a production process as described in WO 2013/135739.

TABLE 2
Composition of the fat component
of IMF-A and IMF-1 (per 100 ml)

| | IMF-A | | IMF-1 | |
|---|---|---|---|---|
| Per 100 ml | Weight | | Weight | |
| Total lipid | 3.5 | g | 3.5 | g |
| Linoleic acid | 454 | mg | 454 | mg |
| Alpha linolenic acid | 54.8 | mg | 54.8 | mg |
| Arachidonic acid | 16.4 | mg | 16.4 | mg |
| Docosahexaenoic acid | 16.5 | mg | 16.5 | mg |
| Palmitic acid | 616 | mg | 616 | mg |
| Milk phospholipids | 52 | mg | 52 | mg |
| Volume-based mode | 0.4 | μm | 3.45 | μm |
| diameter lipid globules | | | | |

| | | |
|---|---|---|
| Volume % of lipid | <10 vol. % | >45 vol. % |
| globules with a diameter | | |
| between 2 and 12 μm | | |
| Coated with phospholipids | Yes | Yes |

### Colonic Microbiota Medium for Fecal Slurry Fermentations:

Yeast extract 1 g/L, Ammoniumsulphate 2 g/L, K₂HPO₄ 2 g/L, NaHCO₃3.2 g/L, NaCl 4.5 g/L, MgSO₄.7H₂O 0.5 g/L, CysteinHCI 0.5 g/L, CaCl₂).2H₂O 0.4 g/L, bile Salts 25 mg/L, 2 ml metal solution (containing per L: 500 mg EDTA, 200 mg FeSO₄.7H₂O, 10 mg ZnSO₄.7H₂O, 3 mg MnCl₂.7H₂O, 30 mg H₃BO₃, 20 mg CoCl₂.6H₂O, 1 mg CuCl₂.2H₂O, 2 mg NiCl₂.6H₂O, 3 mg NaMoO₄.2H₂O, 7.5 mg NaSeO₃), and vitamin solution (containing per L: 1 g menadione, 2 g biotin, 2 g pantothenate, 10 g nicotinamide, 0.5 g cobalamine, 4 g thiamine, 5 g p-aminobenzioc acid; filter-sterilised), and haemin (10 mg/L).

### Methods

Healthy one year old infant stool samples were obtained. The stool samples were fermented in the presence of IMF-A or IMF-1.

The infant fecal samples ware thawed under anaerobic conditions and an approximately 10% (w/v) suspension of fecal sample was made in age adapted Colonic Microbiota medium adjusted to pH 6.5. The diluted fecal sample was homogenized, allowed to sediment for 5 minutes, then filtered over a Millex 100 μm vacuum filter. A Biolector Pro plate (BOH2 round well, M2P-labs) with pH optodes was used. Respectively 2 wells of this plate in row C were filled with 800 μL of the fecal solution and 800 μL of each infant formula (IMFA or IMF1). One feeding row of the plate was filled with sterile 3M NaOH.

After this, the plate was sealed with ventilated silicone foil with slits. The plate was incubated (85% moisture, 37° C., 600 rpm, anaerobic (90% N₂, 5% CO₂, 5% H₂)) in BioLector Pro. The experiment was started with pH at setpoint 6.5 with continuous pH control. After 56 hours of fermentation the experiment was stopped, the fecal slurry of each well was harvested and shortly centrifuged. All under non-septical anaerobic conditions. The supernatant was frozen for further analyses while the fecal pellet was used to extract the DNA prior to 16S rRNA sequencing.

The fecal microbiota was determined by the method as described in Example 1.

### Results

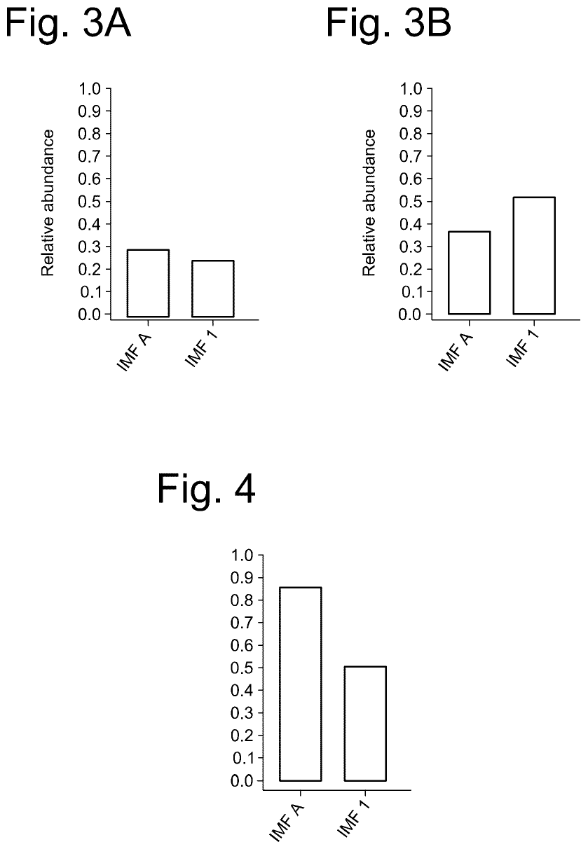

FIGS. 3a-b show the relative abundance of the Enterobacterales and Bifidobacteriaceae, respectively, in the fecal microbiota of the fecal slurry fermented with IMF-A or IMF-1.

FIG. 4 shows the ratio between the relative abundance of Enterobacterales over Bifidobacteriaceae in the fecal microbiota of the fecal slurry fermented with IMF-A or IMF-1.

The order of Enterobacterales is known to encompass opportunistic pathogens, while the family Bifidobacteriaceae is known to encompass beneficial bacteria. It is clear from FIG. 3 that IMF-1 reduced the relative abundance of opportunistic pathogens (FIG. 3a), while increasing the relative abundance of beneficial bacteria (FIG. 3b) compared to IMF-A. FIG. 4 shows that the ratio of opportunistic pathogen to beneficial bacteria is lower for IMF-1 compared to IMF-A. The only difference between IMF-A and IMF-1 is the size of the lipid globules. Therefore, the beneficial effects on the gut microbiota can be ascribed to the presence of the larger lipid globules.

1. A method for improving the gut microbiota in a human infant by treating or preventing gut dysbiosis, the method comprising administering a nutritional composition, selected from infant formula, follow-on formula and young child formula, to the human infant wherein the nutritional composition comprises digestible carbohydrates, protein and lipid, wherein the lipid is in the form of lipid globules, wherein
a. said lipid globules have a volume-weighed mode diameter of at least 1.0 μm; and/or at least 45 vol. %, based on total lipid volume, of the lipid globules have a diameter between 2 and 12 μm; and
b. the lipid comprises 0.5 to 20 wt. % phospholipids based on total lipid and wherein the lipid globules are at least partly coated on the surface with a layer of phospholipids.

2. The method according to claim 1, wherein the improvement of the gut microbiota is by decreasing the relative abundance of opportunistic pathogens in the gut microbiota and/or by increasing the relative abundance of beneficial bacteria in the gut microbiota and/or by decreasing the ratio between the relative abundance of opportunistic pathogens and the relative abundance of beneficial bacteria in the gut microbiota.

3. The method according to claim 2, wherein the opportunistic pathogens are selected from Enterobacterales and Clostridiaceae.

4. The method according to claim 3, wherein the Enterobacterales are Enterobacteriaceae, preferably the Enterobacteriaceae are one of more of the genus Enterobacter, Escherichia, Klebsiella and Shigella.

5. The method according to claim 2, wherein the beneficial bacteria are selected from Lactobacillaceae and/or Bifidobacteriaceae.

6. The method according to claim 1, wherein the use reduces the risk of developing infections in early life.

7. The method according to claim 1, wherein the use reduces the risk of developing allergy.

8. The method according to claim 1, wherein the human infant is at risk of having a compromised gut microbiota.

9. The method according to claim 8, wherein the human infant at risk of having a compromised gut microbiota is selected from the group of infants born via caesarean section, preterm infants, infants born from an overweight or obese mother, infants born from a mother who received antibiotics, infants receiving or having received antibiotics, infants receiving or having received proton pump inhibitors, formula fed infants, or combinations thereof.

10. The method according to claim 1, wherein the phospholipids comprise at least 5 wt. % sphingomyelin based on total phospholipids.

11. The method according to claim 1, wherein the phospholipids are mammalian milk-derived phospholipids.

12. The method according to claim 1, wherein the lipid contains at least 10 wt. % palmitic acid based on total fatty acids and at least 15 wt. % of palmitic acid, based on total palmitic acid, is located at the sn-2 position of a triglyceride.

13. The method according to claim 1, wherein the nutritional composition is for feeding a human infant with an age between 0-36 months.

14. The method according to claim 2, wherein the decrease of the relative abundance of opportunistic pathogens or the increase of the relative abundance of beneficial bacteria in the human infant is compared to an human infant, whom consumed a nutritional composition selected from infant formula, follow-on formula and young child formula, comprising digestible carbohydrates, protein and lipid, wherein the lipid is in the form of lipid globules, wherein
a. the lipid globules have a volume-weighted mode diameter of 0.3-0.5 μm and less than 45 vol. %, based on total lipid volume, of the lipid globules have a diameter above 2 μm; and
the lipid comprises 0.5 to 20 wt. % phospholipids based on total lipid and wherein the lipid globules are at least partly coated on the surface with a layer of phospholipids.
